(19) United States
(12) Patent Application Publication
(10) Pub. No.: US 2026/0103685 A1
(43) Pub. Date: Apr. 16, 2026

(54) A CHROMATOGRAPHY DEVICE, SYSTEM, AND USE THEREOF FOR ANALYTIC SEPARATION

(71) Applicant: Cytiva BioProcess R&D AB, Uppsala (SE)

(72) Inventors: Jean-Luc Maloisel, Uppsala (SE); Åsa Hagner McWhirter, Uppsala (SE); Brigitta Neméth, Uppsala (SE)

(21) Appl. No.: 19/117,286

(22) PCT Filed: Oct. 18, 2023
(86) PCT No.: PCT/EP2023/078920

§ 371 Date: Mar. 31, 2025

(30) Foreign Application Priority Data
Oct. 26, 2022 (GB) 2215814.1

(51) Int. Cl. C12N 7/02; B01D 15/36; B01J 20/285; B01J 20/289

(52) U.S. Cl. CPC C12N 7/02; B01D 15/363; B01J 20/285; B01J 20/289; C12N 2750/14151

(57) ABSTRACT

The present disclosure is directed to a chromatography device for use in an analytic separation method, comprising a chromatography material, which comprises a support and a ligand, wherein the ligand of the chromatography material is defined by the following Formula (I) wherein R₁ is selected from C1-C3 alkyl, and R₂ and R₃ are independently selected from C1-C3 alkyl, CH2OH, and CH2CHOHCH3, wherein the volume of the chromatography material is from about 0.1 mL to about 2 mL. Further disclosed are uses of said chromatography device and a method for separating adeno-associated virus capsids fully packaged with genetic material from adeno-associated virus capsids not fully packaged with genetic material, as well as a kit of parts and a chromatography system for use in analytic separation of adeno-associated capsids.

### TECHNICAL FIELD

The present disclosure relates to the field of analytic separation of adeno-associated capsids and is directed to a chromatography device for use in an analytic separation method, use thereof and a method for separating adeno-associated virus capsids fully packaged with genetic material from adeno-associated virus capsids not fully packaged with genetic material. Further disclosed are a kit of parts and a chromatography system for use in analytic separation of adeno-associated capsids.

### BACKGROUND

Adeno-associated viruses (AAV) are non-enveloped viruses that have linear single-stranded DNA (ssDNA) genome and that can be engineered to deliver DNA to target cells. Recombinant adeno-associated virus (rAAV) vectors have emerged as one of the most versatile and successful gene therapy delivery vehicles. There is an increasing demand to use viral vectors for gene therapy. The AAV vector is one of the most attractive gene transfer tools for developing novel genetic therapies for muscle diseases as well as other disorders. Most of the earlier AAV gene transfer studies used AAV serotype 2 (AAV2). To further improve the efficiency and specificity of AAV-mediated gene transfer, numerous AAV serotypes and variants have been developed by viral genome engineering and/or capsid modification. Use of serotypes like AAV8 and AAV9 have increased in recent years. Target organs determine selection of serotype. To use AAV particles as vectors in therapy it is necessary to purify the virus particles from cell impurities like DNA after transfection. Ultracentrifugation is efficient but not scalable. Normally, several filtration steps and several chromatography steps are used to separate AAV particles from cell cultures (see e.g., Weihong Qu et al, Scalable Downstream Strategies for Purification of Recombinant Adeno-Associated Virus Vectors in Light of the Properties, Current Pharmaceutical Biotechnology 2015 August; 16(8): 684-695).

Therapeutic efficacy of AAV vectors is dependent on high percentage of virus particles fully packaged with genetic material of interest. Upstream expression systems deliver a mixture of fully packaged AAV particles (containing the genetic material of interest), empty AAV particles, and AAV particles which are partially packaged with genetic material of interest), together with impurities. There is thus a need to enrich fully packaged AAV particles in the purification process. However, there are several challenges in relation to achieving an efficient and scalable separation of fully packaged and empty adeno-associated virus capsids, such as:
Large diversity of capsids (serotypes and variants) and cell culture differences in terms of yield of full capsids, which means that extensive optimization is needed for purification of each serotype or variant of adeno-associated virus.
Small differences between fully packaged and empty capsids in relation to several parameters relevant for purification, e.g. isoelectric point;
In addition to fully packaged and empty capsids, also partially packaged capsid variants are produced in the infected host cells. There are indications that such partially packaged, and thereby therapeutically less effective, capsids may be partly co-eluted with fully packaged capsids.

Efficient analytic chromatography materials and analytic separation methods are key to the successful development of large-scale, preparative purification methods. Commercialized analytic products applicable currently used for AAV separation include the monolithic columns CIMac™ AAV full/empty-0.1 Analytical column and CIMmultus™ QA 1 mL Monolithic Column (both from Sartorius, Germany).

However, there is always a need for improved analytic chromatography materials and analytic separation methods for downstream processing of different adeno-associated virus serotypes. Critical issues include obtaining separation between full and empty capsids with a good mass balance to achieve accurate quantitation, giving results comparable to prior art methods like qPCR ELISA and analytic ultracentrifugation. It is further desirable to provide analytic chromatography materials and analytic separation methods applicable to several AAV serotypes, such as AAV2, AAV5, AAV8, and AAV9.

### SUMMARY OF THE DISCLOSURE

The object of the present disclosure is to provide an analytical scale solution providing an improved separation of fully packaged adeno-associated virus capsids from not fully packaged adeno-associated virus capsids. This is achieved by obtaining an improved resolution between fully packaged and not fully packaged capsids by use of a chromatography device as disclosed herein and further when performing an analytic separation method as disclosed herein. The focus of the disclosure is a chromatography device for use in the polishing step, also called secondary or final purification, of an analytic separation method.

More particularly, the present disclosure is directed to a chromatography device for use in an analytic separation method, comprising a chromatography material, which comprises a support and a ligand, wherein the ligand of the chromatography material is defined by the following Formula I:

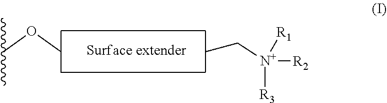

wherein R₁ is selected from C1-C3 alkyl, and R₂ and R₃ are independently selected from C1-C3 alkyl, CH2OH, and CH2CHOHCH3,
wherein the volume of the chromatography material is from about 0.1 mL to about 2 mL.

The present disclosure is also directed to use of said chromatography device for analytic separation of adeno-associated virus capsids fully packaged with genetic material from adeno-associated virus capsids not fully packaged with genetic material present in a liquid sample, wherein the chromatography material has a capacity of separating from about 10¹⁰ to at least about 10¹³ virus capsids, wherein the volume of the liquid sample is from about 70 μL to about 2 mL.

Further disclosed is a method for separating adeno-associated virus capsids fully packaged with genetic material from adeno-associated virus capsids not fully packaged with genetic material present in a liquid sample, comprising the following steps:
a. adding a liquid sample comprising adeno-associated virus capsids of a purity of at least 90% and of a concentration of at least 10¹⁰ adeno-associated virus capsids/mL, of which at least 10% of the adeno-associated virus capsids are adeno-associated virus capsids fully packaged with genetic material, to a chromatography material comprised by the herein disclosed chromatography device;
b. obtaining the adeno-associated virus capsids not fully packaged with genetic material in a flow-through mode, or eluting the adeno-associated virus capsids not fully packaged with genetic material from the chromatography material;
c. eluting the adeno-associated virus capsids fully packaged with genetic material from the chromatography material;
wherein the adeno-associated virus capsids eluted in step (c) are eluted into eluate fractions, which eluate fractions combined comprise at least 80% of the adeno-associated virus capsids of the liquid sample added in step (a), of which at least 90% of the adeno-associated virus capsids are fully packaged with genetic material.

The present disclosure is also directed to a kit of parts for analytical separation of adeno-associated virus capsids fully packaged with genetic material from adeno-associated virus capsids not fully packaged with genetic material present in a liquid sample, the kit of parts comprising the herein disclosed chromatography device, one or more buffers, and optionally a computer program product directly loadable into the internal memory of a digital computer, which computer program product comprises software code means for translation of UV signal ratio into viral capsids per mL.

Further disclosed is a chromatography system comprising:
a. a sample loop having a volume of from about 10 μL to about 2 mL;
b. the herein disclosed chromatography device;
c. a detector for measuring absorbance, optionally for measuring absorbance at 260 nm and 280 nm;
d. a detector for measuring fluorescence, optionally for measuring excitation at 280 nm and emission at 348 nm;
e. a detector for measuring multi-angle light scattering, optionally at an angle of 90°;
f. pumps for pumping flows and volumes relevant for analytic separation of adeno-associated virus capsids fully packaged with genetic material from adeno-associated virus capsids not fully packaged with genetic material present in a liquid sample;
and optionally
g. a computer program product directly loadable into the internal memory of a digital computer, which computer program product comprises software code means for translation of UV signal ratio into viral capsids per mL.

In particular, the present disclosure is directed to separation of adeno-associated virus capsids of adeno-associated virus serotypes 1, 2, 4, 5, 6, 7, 8, 9, and 10 (AAV1, AAV2, AAV4, AAV5, AAV6, AAV7, AAV8, AAV9, and AAV10) or a variant thereof.

Preferred aspects of the present disclosure are described below in the detailed description and in the dependent claims.

### BRIEF DESCRIPTION OF THE DRAWINGS

FIG. 1 is a flow chart of a method for separating adeno-associated virus capsids according to the present disclosure.

FIG. 2 A-B are graphs showing the elution curves for fully packaged and empty AAV8 capsids by use of the herein disclosed chromatography device as described in the Example below.

FIG. 3 A-B are graphs showing the elution curves for fully packaged and empty AAV9 capsids by use of the herein disclosed chromatography device as described in the Example below.

FIG. 4 is a graph showing the elution curves for fully packaged and empty AAV8 capsids by use of a previously known chromatography device as described in the Example below.

FIG. 5 is a graph showing the elution curve for fully packaged and empty AAV5 capsids by use of a previously known chromatography device as described in the Example below.

### DETAILED DESCRIPTION OF THE DISCLOSURE

The present disclosure solves or at least mitigates the problems associated with existing chromatography materials and devices for analytic separation, by providing a chromatography device for use in an analytic separation method, comprising a chromatography material, which comprises a support and a ligand, wherein the ligand of the chromatography material is defined by the following Formula I:

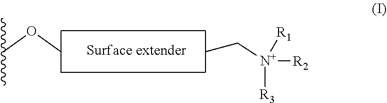

wherein R₁ is selected from C1-C3 alkyl, and R₂ and R₃ are independently selected from C1-C3 alkyl, CH2OH, and CH2CHOHCH3,
wherein the volume of the chromatography material is from about 0.1 mL to about 2 mL.

As a non-limiting example, each of R₁, R₂, and R₃ is CH3.

There are currently available chromatography materials comprising a ligand defined by Formula I, wherein each of R₁, R₂, and R₃ is CH3; e.g., a chromatography material made available under the name Capto Q, provided by Cytiva, Sweden (www.cytivalifesciences.com). Capto Q further comprises dextran as surface extender and is a chromatography medium for high-resolution polishing steps in industrial purification processes, e.g., for purification of monoclonal antibodies. However, previously it has not been used for analytic separation of fully packaged adeno-associated capsids from not fully packaged adeno-associated capsids. Capto Q is a strong anion exchange chromatography material having about 100% quaternized amine groups. Herein, the term “strong anion exchange chromatography material” is intended to mean a chromatography material which comprises a ligand comprising a quaternized amine group. A quaternary amine group is a strong anion exchange group, which is always positively charged irrespective of to which pH it is subjected. A degree of quaternization of the amine group of from about 12% to about 100% globally in a chromatography material is generally considered to result in a chromatography material which behaves like a strong, or at least partially strong, anion exchange chromatography material since these at least 12% of all amine groups are always charged. In contrast to quaternized amine groups, almost all other ionic exchange groups are weak, i.e., their charge varies from fully charged to not charged within a reasonable range of pH used (such as pH 2-11) and having a neutral charge (same amount of + and − charges) at pl.

According to another non-limiting example, R₁ and R₂ are ethyl, and R₃ is methyl.

According to yet another non-limiting example, R₁ and R₂ are methyl, and R₃ is CH2CHOHCH3.

In a currently preferred embodiment, the chromatography device is for use in an analytic method for separation of adeno-associated virus capsids fully packaged with genetic material from adeno-associated virus capsids not fully packaged with genetic material.

Significant advantages of the presently disclosed analytic chromatography device include that it achieves improved resolution between full and empty AAV capsids compared to currently available analytic chromatography devices, as shown in the Examples herein. Further, this analytic product is scalable all the way up to large-scale purification of AAV and it is applicable to all AAV serotypes. It may thus be used as a process development tool for screening of optimal separation conditions for different AAV serotypes. It is particularly aimed for use in the polishing step of a purification method. The presently disclosed chromatography device is also versatile in the sense that it can be used together with different currently available purification systems.

A “virus particle” is herein used to denote a complete infectious virus particle. It includes a core, comprising the genome of the virus (i.e., the viral genome), either in the form of ribonucleic acid (RNA) or deoxyribonucleic acid (DNA), and the core is surrounded by a morphologically defined shell. The shell is called a capsid. The capsid and the enclosed viral genome together constitute the so-called nucleocapsid. The nucleocapsid of some viruses is surrounded by a lipoprotein bilayer envelope. In the field of bioprocessing, for the purpose of producing viral vectors for various applications such as therapy, the genome of a virus particle is modified to include a genetic insert, comprising genetic material of interest. Modified virus particles are allowed to infect host cells in a cell culture and the virus particles are propagated in said host cells, after which the virus particles are purified from the cell culture by any means of separation and purification. Herein, a virus particle to be separated from a cell culture by the presently disclosed method may alternatively be referred to as a “target molecule” or “target”. It is to be understood that “a virus particle” is intended to mean a type of virus particle and that the singular form of the term may encompass a large number of individual virus particles. Herein, the term “virus particle” may be used interchangeably with the terms “vector” and “capsid”, respectively, as further defined below.

The term “vector” is herein used to denote a virus particle, normally a recombinant virus particle, which is intended for use to achieve gene transfer to modify specific cell type or tissue. A virus particle can for example be engineered to provide a vector expressing therapeutic genes. Several virus types are currently being investigated for use to deliver genetic material (e.g., genes) to cells to provide either transient or permanent transgene expression. These include adenoviruses, retroviruses (γ-retroviruses and lentiviruses), poxviruses, adeno-associated viruses (AAV), baculoviruses, and herpes simplex viruses. Herein, the term “vector” may be used interchangeably with the terms “virus particle” and “capsid”, respectively.

The term “capsid” means the shell of a virus particle. The capsid surrounds the core of the virus particle, wherein the core normally should comprise a viral genome. A modified (recombinant) capsid, as produced in an upstream process of manufacturing, is supposed to comprise a complete viral genome, which genome includes genetic material of interest for one or more applications, for example of interest for various therapeutic applications. However, owing to low packaging efficiency, assembled capsids do not always contain any genetic material or only encapsidate truncated genetic fragments, resulting in so-called empty capsids and partially filled capsids, respectively. These capsids possess no therapeutic function, yet they compete for binding receptors during the cell-mediated processes. This may diminish the overall therapeutic efficacy and trigger undesirable immune responses. As a result, tracking these capsids throughout the production process is crucial to ensure consistent product quality and a proper dosing response (Xiaotong Fu et al, Analytical Strategies for Quantification of Adeno-Associated Virus Empty Capsids to Support Process Development, Human gene therapy methods, 2019, 30(4): 144-152). In up to 20-30% of a population of virus particles artificially produced in a cell culture, the capsid is only partially filled with genetic material. Further, in up to as much as 98% of artificially produced virus particles, the capsid does not comprise any part of the viral genome at all, i.e., it is empty. However, generally between 80% to 90% of artificially produced virus particles have empty capsids, and best cases currently achieve as little as 50% empty capsids.

Herein, the term “capsid” may be used interchangeably with the terms “vector” and “virus particle”, respectively. In the context of the present disclosure, a capsid may or may not comprise genetic material.

The term “genetic material of interest” is intended to mean genetic material which in the field of bioprocessing is considered relevant and valuable to get produced by viral replication and to purify such that it can be used in various applications, such as, but not limited to, therapeutic applications. As a non-limiting example, genetic material of interest may comprise a therapeutically relevant genetic material, such as a therapeutically relevant nucleotide sequence.

The term “capsid fully packaged with genetic material” is herein used to denote a capsid which has been correctly produced (by the host cell), or in other words,
a capsid which comprises a complete viral genome, or in other words,
a capsid comprising 100% of its viral genome, or in other words,
a capsid comprising a functional viral genome.

The viral genome includes a genetic insert, comprising genetic material of interest, as defined elsewhere herein.

A capsid which comprises a complete viral genome may herein alternatively be called a “full capsid” or a “fully packaged capsid”. The terms “full capsid”, “fully packaged capsid”, and “capsid fully packaged with genetic material” may be used interchangeably throughout this text.

The term “capsid not fully packaged with genetic material” is herein used to denote a capsid which has not been correctly produced (by the host cell), or in other words,
a capsid which does not comprise a complete viral genome, or in other words,
a capsid which comprises less than 100% of its viral genome.

A capsid which is not fully packaged with genetic material is either partially filled with genetic material or is not filled with any genetic material at all.

The term “capsid not fully packaged with genetic material” encompasses the terms “partially filled capsid” and “empty capsid”, as defined below.

A “partially filled capsid” is herein defined as a capsid which comprises parts of its viral genome, such as defective parts of its viral genome, or in other words,
a capsid which comprises a partial viral genome, or in other words,
a capsid which comprises a non-complete viral genome, or in other words,
a capsid which comprises a defective viral genome, or in other words,
a capsid which comprises more than 0% and less than 100% of the complete viral genome, such as from about 1% to about 99%, such as from about 5% to about 95%, such as from about 10% to about 90%, or such as about 1%, about 5%, about 10%, about 20%, about 30%, about 40%, about 50%, about 60%, about 70%, about 80%, about 90%, about 95%, or about 99%, of the complete viral genome. Since a partially filled capsid is an incorrectly produced capsid, it is desirable to separate and remove as many as possible of the partially filled capsids from a population of capsids, before putting the population of capsids to use in its intended application, e.g., a therapeutic application. Herein, a partially filled capsid may alternatively be called an “intermediate capsid”.

An “empty capsid” is herein defined as a capsid which does not comprise any part of its viral genome, i.e., which comprises 0% of its viral genome, or in other words,
a capsid which is not filled with any genetic material at all. Thus, an empty capsid does not comprise any genetic material of interest. Consequently, it is desirable (and sometimes required, e.g., due to clinical regulations) to separate and remove as many as possible of the empty capsids from a population of capsids, before putting the population of capsids to use in its intended application, e.g., a therapeutic application.

Before putting a population of virus particles to use in its intended application, e.g., a therapeutic application, it is desirable (sometimes even required, e.g., due to clinical regulations) to enrich the full capsids, i.e., to increase the percentage of full capsids at the expense of the percentage of partially filled capsids and empty capsids.

The percentage of full capsids and empty capsids in a population of capsids can be estimated or analyzed with several methods known in the art. Some of these methods are briefly described below:
1: A260:280 in chromatogram will give an estimation of percentage full capsids present in peaks (ratio 1-1.5 indicate enriched in full capsids, ratio 0.5-0.7 is containing mainly empty capsids).
2. qPCR:ELISA ratio. qPCR quantifies viral genomes and ELISA quantifies total viral particles. A ratio of 2 assays with variation is less accurate and will be uncertain. Requires orthogonal analysis for confirmation (see below, 3, 4 or 5).
3. Analytical anion exchange separating full and empty capsids (A260:280 ratio and peak area to calculate the percentage). Accuracy dependent of peak definition.
4. Analytical ultracentrifugation (AUC). Detects and quantifies particles of different density (corresponding to full, partially filled, and empty capsids). This is currently known as the “golden standard” in the art. However, ultracentrifugation is not scalable and thus is not suitable for analysis of large-scale batches of capsids.
5. Transmission electron microscopy (TEM). Image analysis counting particles (full, partially filled, and empty capsids). May introduce artifacts from sample preparation.

Some methods for estimating or analyzing the percentage of full capsids and empty capsids in a population of capsids are described in more detail in Xiaotong Fu et al, Analytical Strategies for Quantification of Adeno-Associated Virus Empty Capsids to Support Process Development, Human gene therapy methods, 2019, 30(4): 144-152, which is hereby incorporated by reference herein.

The term “separation matrix” is used herein to denote a material comprising a support to which one or more ligands comprising functional groups have been coupled. The functional groups of the ligand(s) bind compounds herein also called analytes, which are to be separated from a liquid sample and/or which are to be separated from other compounds present in the liquid sample. A separation matrix may further comprise a compound which couples the ligand(s) to the support. The terms “linker”, “extender”, and “surface extender” may be used to describe such a compound, as further described below. The term “resin” is sometimes used for a separation matrix in this field. The terms “chromatography material” and “chromatography matrix” are used herein to denote a type of separation matrix.

The term “surface” herein means all external surfaces and includes in the case of a porous support outer surfaces as well as pore surfaces.

The separation matrix may be contained in any type of separation device. The term “separation device” has its conventional meaning in the field of bioprocessing and is to be understood as encompassing any type of separation device which is capable of and suitable for separating and purifying compounds from a fluid containing by-products from the production of the compounds.

The term “chromatography device” is used herein to denote a type of separation device.

Non-limiting examples of separation devices suitable for use according to the present disclosure include chromatography columns and membrane devices. Such separation devices may suitably comprise chromatography material comprising a ligand as defined by Formula I, as described in detail elsewhere herein.

In this context, “ligand” is a molecule that has a known or unknown affinity for a given analyte and includes any functional group, or capturing agent, immobilized on its surface, whereas “analyte” includes any specific binding partner to the ligand. The term “ligand” may herein be used interchangeably with the terms “specific binding molecule”, “specific binding partner”, “capturing molecule” and “capturing agent”. Herein, the molecules in a liquid sample which interact with a ligand are referred to as “analyte”. The analytes of interest according to the present disclosure are adeno-associated virus capsids, more particularly adeno-associated virus capsids either fully packaged or not fully packaged with genetic material. Consequently, herein the terms “analyte”, “adeno-associated virus capsid” and “capsid” may be used interchangeably.

The chromatography material herein disclosed comprises a support to which the ligand is coupled. The support may be made from an organic or inorganic material and may be porous or non-porous. In one embodiment, the support is prepared from a native polymer, such as cross-linked carbohydrate material, e.g. agarose, agar, cellulose, dextran, chitosan, konjac, carrageenan, gellan, alginate, pectin, starch, etc. The native polymer supports are easily prepared and optionally cross-linked according to standard methods, such as inverse suspension gelation (S Hjerten: Biochim Biophys Acta 79(2), 393-398 (1964). In an especially advantageous embodiment, the support is a kind of relatively rigid but porous agarose, which is prepared by a method that enhances its flow properties, see e.g. U.S. Pat. No. 6,602,990 (Berg). In an alternative embodiment, the support is prepared from a synthetic polymer or copolymer, such as cross-linked synthetic polymers, e.g. styrene or styrene derivatives, divinylbenzene, acrylamides, acrylate esters, methacrylate esters, vinyl esters, vinyl amides etc. Such synthetic polymers are easily prepared and optionally cross-linked according to standard methods, see e.g. “Styrene based polymer supports developed by suspension polymerization” (R Arshady: Chimica e L'Industria 70(9), 70-75 (1988)). Native or synthetic polymer supports are also available from commercial sources, such as Cytiva, Sweden, for example in the form of porous particles. In yet an alternative embodiment, the support is prepared from an inorganic polymer, such as silica. Inorganic porous and non-porous supports are well known in this field and easily prepared according to standard methods.

The support of the chromatography material may be in the form of particles, such as substantially spherical, elongated or irregularly formed particles.

Where the chromatography material is in the form of particles, the particles may be particles having a homogeneous porosity and being at least partly permeable to adeno-associated virus capsids.

Herein, the term “homogeneous porosity” is intended to mean that a particle having a homogeneous porosity has a homogeneous porosity throughout its entire structure or volume, such that each particle is at least partly permeable to adeno-associated virus capsids throughout its entire structure or volume. In other words, a particle having a homogeneous porosity has a porosity which permits adeno-associated virus capsids to diffuse, completely or at least partly, through its pores, throughout the entire structure or volume of the particle.

Adeno-associated viruses are approx. 20-25 nm in diameter. Since a capsid is the shell of a virus particle, and since adeno-associated viruses do not have a lipoprotein bilayer envelope surrounding the capsid, the size of an adeno-associated virus capsid is approx. 20-25 nm in diameter.

Accordingly, where the chromatography material is in the form of particles having a homogeneous porosity and being at least partly permeable to adeno-associated virus capsids, each particle may suitably comprise pores of a diameter which is >25 nm, i.e., larger than the diameter of the adeno-associated virus capsids to be separated, thereby enabling diffusion of capsids within the entire particle. It is to be understood that for the specific purposes of the present disclosure, i.e., to separate adeno-associated virus capsids, a diameter >25 nm may be of any size >25 nm, including but not limited to 30, 50, 75, 100, 150, or 200 nm.

Further, it is to be understood that a particle having a homogeneous porosity throughout its entire structure or volume nevertheless may comprise pores of different sizes, both pores that are large enough to easily allow capsids to diffuse within the particle and pores that are small enough not to allow diffusion of capsids. This diversity of pore size can be measured by the diffusion coefficient of a molecule of a well-defined molecular weight and hydrodynamic size. As a non-limiting example, dextran, which has a molecular weight of 140-225 kDa or a hydrodynamic diameter of 20-25 nm (i.e., a diameter of the same size as adeno-associated virus capsids), can be used to evaluate the degree of diffusion of adeno-associated virus capsids within the pores of the particles.

The chromatography material Capto Q, advantageously used in the Example herein, comprise a support in the form of substantially spherical particles or beads, which have a diameter of approx. 90 μm. This type of particle is a non-limiting example of a particle having a homogeneous porosity (i.e., throughout its entire structure or volume) and being at least partly permeable to adeno-associated virus capsids (i.e., throughout its entire structure or volume).

Suitable particle sizes of a chromatography material for use in the presently disclosed methods may be in a diameter range of 5-500 μm, such as 10-100 μm, e.g., 30-90 μm. In the case of essentially spherical particles, the average particle size may be in the range of 5-1000 μm, such as 10-500. In a specific embodiment, the average particle size is in the range of 10-200 μm. The skilled person in this field can easily choose the suitable particle size and porosity depending on the process to be used. For example, for a large-scale process, for economic reasons, a more porous but rigid support may be preferred to allow processing of large volumes, especially for the capture step. In chromatography, process parameters such as the size and the shape of the column will affect the choice. In an expanded bed process, the matrix commonly contains high density fillers, preferably stainless-steel fillers. For other processes other criteria may affect the nature of the matrix.

The chromatography material may be dried, such as dried particles which upon use are soaked in liquid to retain their original form. For example, such a dried chromatography material may comprise dried agarose particles.

The chromatographic support material may alternatively take other shapes conventionally used in separation, such as monoliths, filters, membranes, or nanofibers, etc., wherein a volume of a chromatography material as disclosed herein is applicable also to such shapes of the support.

Equally applicable to the concept of the present disclosure, but where other features than a specific volume of the chromatography material disclosed herein may be useful to achieve the same purpose, are other supports conventionally used for analytics, such as capillaries, chips or surfaces.

Where the support of the chromatography material comprises a monolith, a suitable pore diameter in the monolith for the purpose of separating adeno-associated virus capsids ranges from a minimum pore diameter of >25 nm, i.e., larger than the diameter of the capsids to be separated, and up to a maximum pore diameter of about 5 μm, such as about 0.5, 1.0, 2.0, 3.0, 4.0, or 5.0 m.

Where the support of the chromatography material comprises nanofibers, such nanofibers may for example comprise electrospun polymer nanofibers. When in use, such nanofibers form a stationary phase comprising a plurality of pores through which a mobile phase can permeate.

The support of the chromatography material may comprise a membranous structure, such as a single membrane, a pile of membranes or a filter. The membrane may be an adsorptive membrane. Where the support of the chromatography material comprises a membranous structure, a suitable pore diameter in the membranous structure for the purpose of separating adeno-associated virus capsids ranges from a minimum pore diameter of >25 nm, i.e., larger than the diameter of the capsids to be separated, and up to a maximum pore diameter of about 5 μm, such as about 0.5, 1.0, 2.0, 3.0, 4.0, or 5.0 μm. Where the chromatography material comprises a membranous structure, such membranous structure may for example comprise a nonwoven web of polymer nanofibers.

Non-limiting examples of suitable polymers useful for the chromatographic support material may be selected from polysulfones, polyamides, nylon, polyacrylic acid, polymethacrylic acid, polyacrylonitrile, polystyrene, and polyethylene oxide, and mixtures thereof.

Alternatively, the polymer may be a cellulosic polymer, such as selected from a group consisting of cellulose and a partial derivative of cellulose, particularly cellulose ester, cross-linked cellulose, grafted cellulose, or ligand-coupled cellulose. Cellulose fiber chromatography (known as Fibro chromatography; Cytiva, Sweden) is an ultrafast chromatography purification for short process times and high productivity, which utilizes the high flow rates and high capacities of cellulose fiber. Where the support of the chromatography material comprises cellulose fibers such as Fibro, a suitable pore diameter in the cellulose fiber for the purpose of separating adeno-associated virus capsids ranges from a minimum pore diameter of >25 nm, i.e., larger than the diameter of the capsids to be separated, and up to a maximum pore diameter of about 5 μm, such as about 0.1, 0.2, 0.5, 1.0, 2.0, 3.0, 4.0, or 5.0 m.

The term “membrane chromatography” has its conventional meaning in the field of bioprocessing. In membrane chromatography there is binding of components of a fluid, for example individual molecules, associates or particles, to the surface of a solid phase in contact with the fluid. The active surface of the solid phase is accessible for molecules by convective transport. The advantage of membrane adsorbers over packed chromatography columns is their suitability for being run with much higher flow rates. This is also called convection-based chromatography. A convection-based chromatography matrix includes any matrix in which application of a hydraulic pressure difference between the inflow and outflow of the matrix forces perfusion of the matrix, achieving substantially convective transport of substance(s) into the matrix or out of the matrix, which is effected very rapidly at a high flow rate. Convection-based chromatography and membrane adsorbers are described in for example US20140296464A1, US20160288089A1, WO2018011600A1, WO2018037244A1, WO2013068741A1, WO2015052465A1, U.S. Pat. No. 7,867,784B2, hereby incorporated by reference in their entirety.

In the herein disclosed chromatography device, the chromatography material used comprises a linker connecting the ligand to the support, i.e., the coupling of the ligand to the support is provided by introducing a linker between the support and ligand. The coupling may be carried out following any conventional covalent coupling methodology such as by use of epichlorohydrin; epibromohydrin; allyl-glycidylether; bis-epoxides such as butanedioldiglycidylether; halogen-substituted aliphatic substances such as di-chloro-propanol; and divinyl sulfone. Other non-limiting examples of suitable linkers are: polyethylene glycol (PEG) having 2-6 carbon atoms, carbohydrates having 3-6 carbon atoms, and polyalcohols having 3-6 carbon atoms. These methods are all well known in the art and easily carried out by the skilled person.

The ligand is preferably coupled to the support via a longer linker molecule, also known as a “surface extender”, or simply “extender”. Extenders are well known in this field, and commonly used to sterically increase the distance between ligand and support. Extenders are sometimes denoted tentacles or flexible arms. For a more detailed description of possible chemical structures, see for example U.S. Pat. No. 6,428,707, which is hereby included herein by reference. In brief, the extender may be in the form of a polymer such as a homo- or a copolymer. Hydrophilic polymeric extenders may be of synthetic origin, i.e., with a synthetic skeleton, or of biological origin, i.e., a biopolymer with a naturally occurring skeleton. Typical synthetic polymers are polyvinyl alcohols, polyacryl- and polymethacrylamides, polyvinyl ethers etc. Typical biopolymers are polysaccharides, such as starch, cellulose, dextran, agarose.

The volume of the chromatography material disclosed herein may be from about 0.1 mL to about 2 mL, such as from about 0.1 mL to about 1.5 mL, such as about 0.1 mL to about 1.0 mL or about 0.1 mL to about 0.5 mL, such as 0.1 mL, 0.2 mL, 0.3 mL, 0.4 mL, 0.5 mL, 0.6 mL, 0.7 mL, 0.8 mL, 0.9 mL, 1.0 mL, 1.1 mL, 1.2 mL, 1.3 mL, 1.4 mL, or 1.5 mL.

How to determine a volume of a chromatography material is well-known to the skilled person. As an example, when the support of the chromatography material is in the form of a resin and comprises particles, the volume of the chromatography material is the volume of the resin present in the chromatography device when the resin has been allowed to sediment or settle to its target bed height/packed bed volume, or similarly, at the correct level of compression. Naturally, the volume of the chromatography material, when in the form of spherical particles and when present in the chromatography device, may comprise spaces between them and may also be porous, all of which is also included in the term volume of a chromatography material. The time needed to allow the resin to settle may vary depending on factors such as the starting material used, e.g. the resin slurry concentration and choice of buffers. However, all of these are choices of standard methods conventionally used in the field of separation methods based on chromatography.

The chromatography material comprised by the herein disclosed chromatography device has a capacity of separating from about 10¹⁰ to at least about 10¹³ virus capsids, such as about 10¹⁰, 10¹¹, 10¹², 10¹³, or 10¹⁴ virus capsids virus capsids from a liquid sample having a volume of from about 70 μL to about 2 mL, such as a volume of about 70 μL, 80 μL, 90 μL, 100 μL, 200 μL, 300 μL, 400 μL, 500 μL, 600 μL, 700 μL, 800 μL, 900 μL, 1 mL, 1.25 mL, 1.5 mL, 1.75 mL, or 2 mL.

It is to be understood that the term “liquid sample” as used herein encompasses any type of sample obtainable from a cell culture, or from a fluid originating from a cell culture which fluid is at least partly purified, by any means of separation and purification.

The density of ligand defined by Formula I may be from about 60 to about 500 μmol, such as from about 160 to about 350 μmol, such as from about 160 to about 220 mol, of ligand per ml of the chromatography material.

As described above, the chromatography material comprises a surface extender connecting the ligand to the support, wherein the surface extender is a polymer, wherein the polymer is selected from:
(i) a polymer having a naturally occurring skeleton, such as a polysaccharide, such as starch, cellulose, dextran, or agarose; and
(ii) a polymer having a synthetic skeleton, such as a polyvinyl alcohol, a polyacrylamide, a polymethacrylamide, or a polyvinyl ether.

As a non-limiting example, the surface extender is dextran. The dextran may have a molecular weight of from about 10 to about 2000 kDa, such as about 10, 40, 70, 250, 750, or 2000 kDa, such as 40 kDa. The density of dextran may be from about 5 to about 30 mg dextran per ml of the chromatography material. It is to be understood that the amount of dextran immobilized on the chromatography material may vary, for example depending on the molecular weight of the dextran immobilized. Normally, decreasing amounts are required for increasing molecular weights of dextran.

The present disclosure is directed to use of the herein disclosed chromatography device for analytic separation of adeno-associated virus capsids fully packaged with genetic material from adeno-associated virus capsids not fully packaged with genetic material present in a liquid sample, wherein the chromatography material has a capacity of separating from about 10¹⁰ to at least about 10¹³ virus capsids, wherein the volume of the liquid sample is from about 70 μL to about 2 mL.

Advantageously, said use of the chromatography device may require a shorter time for separating full capsids from empty capsids compared to prior art chromatography devices, as shown in the Example herein. More particularly, the minimum time for the liquid sample to pass through the chromatography material may be from about 2 minutes to about 5.5 minutes, such as about 5.5 minutes, 5 minutes, 4.5 minutes, 4 minutes, 3.5 minutes, 3 minutes, 2.5 minutes, or 2 minutes. However it is contemplated that also longer run times may be useful.

Further, said use of the chromatography device is applicable to adeno-associated virus capsids of different serotypes, such as capsids of adeno-associated virus serotype 1 (AAV1), adeno-associated virus serotype 2 (AAV2), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), or adeno-associated virus serotype 10 (AAV10), or a variant thereof. In a currently preferred embodiment of said use, the adeno-associated virus capsids are capsids of adeno-associated virus serotype 9 (AAV9) or a variant thereof.

The term “variant” in relation to an adeno-associated virus (AAV) serotype 1, 2, 4, 5, 6, 7, 8, or 10, as listed above, is intended to mean a modified or engineered AAV, in which the capsid structure has been modified to improve clinical performance, for example towards a specific target organ. As a non-limiting example, an AAV8 variant comprises capsid parts of AAV8 and may additionally comprise capsid parts of other AAV serotypes than AAV8, such as AAV5. However, an AAV8 variant as referred to herein must retain a significant structural similarity to a non-modified AAV8 capsid, such as retaining at least 50%, such as 60%, 70%, 80%, or 90%, of the external surface structure of a non-modified AAV8 capsid. This applies equally to a variant of AAV serotype 1, 2, 4, 5, 6, 7, or 10, as compared to a non-modified AAV serotype 1, 2, 4, 5, 6, 7, or 10, respectively. Further, as a non-limiting example, in the context of purification or separation of a variant of AAV8, a “variant” is herein defined as an adeno-associated virus which has a functionally equivalent binding capacity to the ligand of a specified chromatography material, compared to the binding capacity of the original AAV8 to said specified chromatography material. This applies equally to a variant of AAV serotype 1, 2, 4, 5, 6, 7, or 10, as compared to the original AAV serotype 1, 2, 4, 5, 6, 7, or 10, respectively. The specified chromatography material may, for example, be the chromatography material as disclosed in more detail elsewhere herein. A variant of an adeno-associated virus may for example be obtained by spontaneous mutation, or by engineered modification (i.e., obtained by human interaction), of one or more nucleotides of the genome of the adeno-associated virus.

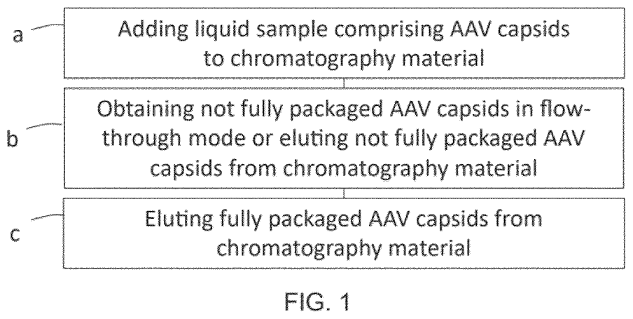

The present disclosure further solves or at least mitigates the problems associated with existing analytic methods for separating full and empty AAV capsids by providing, as illustrated in FIG. 1, a method for separating adeno-associated virus capsids fully packaged with genetic material from adeno-associated virus capsids not fully packaged with genetic material, the method comprising the following steps:
a. adding a liquid sample comprising adeno-associated virus capsids of a purity of at least 90% and of a concentration of at least 10¹⁰ adeno-associated virus capsids/mL, of which at least 10% of the adeno-associated virus capsids are adeno-associated virus capsids fully packaged with genetic material, to a chromatography material comprised by the herein disclosed chromatography device, as described in detail elsewhere herein;
b. obtaining the adeno-associated virus capsids not fully packaged with genetic material in a flow-through mode, or eluting the adeno-associated virus capsids not fully packaged with genetic material from the chromatography material;
c. eluting the adeno-associated virus capsids fully packaged with genetic material from the chromatography material;
wherein the adeno-associated virus capsids eluted in step (c) are eluted into eluate fractions, which eluate fractions combined comprise at least 80% of the adeno-associated virus capsids of the liquid sample added in step (a), of which at least 90% of the adeno-associated virus capsids are fully packaged with genetic material.

The volume of the liquid sample added in step (a) may be from about 70 μL to about 2 mL, such as a volume of about 70 μL, 80 μL, 90 μL, 100 μL, 200 μL, 300 μL, 400 μL, 500 μL, 600 μL, 700 μL, 800 μL, 900 μL, 1 mL, 1.25 mL, 1.5 mL, 1.75 mL, or 2 mL.

Significant advantages of the presently disclosed method include that it provides an improved resolution between full and empty AAV capsids in a shorter time compared to prior art analytic separation methods. More particularly, steps (a)-(c) and any intermediate steps can be completed within 5.5 minutes, such as 5.5 minutes, 5 minutes, 4.5 minutes, 4 minutes, 3.5 minutes, 3 minutes, 2.5 minutes, or 2 minutes.

The term “eluent” is used in its conventional meaning in this field, i.e., a buffer of suitable pH and/or ionic strength to release one or more compounds from a separation matrix.

The term “eluate” is used in its conventional meaning in this field, i.e., the part(s) of a liquid sample which are eluted from a chromatography column after having loaded the liquid sample onto the chromatography column.

As mentioned above, in the method for separating fully packaged capsids from not fully packaged capsids, the liquid sample which is added to a chromatography material in step (a) comprises adeno-associated virus capsids of a purity of at least 90%, such as 90%, 91%, 92%, 93%, 94%, 95%, 96%, 97%, 98%, or 99% and of a concentration of at least 10¹⁰, such as 10¹¹, 10¹², 10¹³, 10¹⁴, or 10¹⁵, adeno-associated virus capsids/ml, of which at least 10%, such as 15%, 20%, 25%, 30%, 40%, 50%, 60%, 70%, or 80%, of the adeno-associated virus capsids are adeno-associated virus capsids fully packaged with genetic material. With regard to the purity of adeno-associated capsids in the liquid sample, a purity of at least 90%, such as up to 99%, is intended to mean that at least 90%, such as up to 99%, of the biological material in the liquid sample is represented by adeno-associated capsids (including full, empty, and partially filled capsids) while the remaining up to 10%, such as 1%, is represented by host cell protein and DNA.

The aim of steps (b) and (c) of the above-disclosed method is to obtain fully packaged capsids of a purity which is as high as possible. A person skilled in the art readily understands that this may be achieved by applying various different separation conditions. Non-limiting examples of separation conditions to obtain fully packaged capsids of a purity as high as possible include separation conditions which allow binding of fully packaged capsids to the chromatography material, while:
(i) allowing not fully packaged capsids to substantially flow through the chromatography material (i.e., not fully packaged capsids substantially not binding to the chromatography material), or
(ii) allowing not fully packaged capsids to bind to the chromatography material followed by eluting them from the chromatography material. It is to be understood that in the bind-elute process described in item (ii), the not fully packaged capsids may be eluted from the chromatography material before or after fully packaged capsids, depending on which separation conditions are applied.

As mentioned above, there are small differences between fully packaged capsids and not fully packaged capsids in relation to several parameters relevant for purification, e.g., their isoelectric point. This often leads to (at least partial) co-elution of fully packaged and not fully packaged capsids. Accordingly, realistically, the adeno-associated virus capsids eluted in steps (b) and (c) of the above-disclosed method will not be completely separated into full, empty, and partially filled capsids. However, there will be eluate fractions which comprise a substantially higher percentage of full capsids than in the liquid sample added to the chromatography material in step (a). More particularly, as disclosed above, the adeno-associated virus capsids eluted in step (c), i.e., adeno-associated virus capsids fully packaged with genetic material, are eluted into eluate fractions, which eluate fractions combined comprise at least 80%, such as 85%, or 90% of the adeno-associated virus capsids of the liquid sample added in step (a), of which at least 90%, such as 95% of the adeno-associated virus capsids are fully packaged with genetic material.

Steps (a), (b), and (c) of the above-disclosed method may comprise applying a buffer having a pH of from about 6.0 to about 10.5, such as from about 7.0 to about 10.0, such as from about 7.5 to about 9.5, or about 6.0, 6.5, 7.0, 7.5, 8.0, 8.5, 9.0, 9.5, 10.0, or 10.5. According to a non-limiting example, as described in the Example below, a pH of about 9.0 may be applied for the herein disclosed chromatography material.

Said buffer is suitably selected from buffers generally recommended for anion exchange chromatography and may for example comprise tris(hydroxymethyl)amino-methane (i.e., Tris), 1,3-bis(tris(hydroxymethyl)methylamino) propane (i.e., bis-Tris propane), triethanolamine, N-methyldiethanolamine, Diethanolamine, 1,3-diaminopropane, or ethanolamine. A person skilled in the art is able to choose a suitable concentration for any one of the above-listed buffers.

In the above-disclosed method, step (c), and step (b) when not fully packaged capsids are bound to the chromatography material, may comprise applying a buffer, optionally one of the buffers mentioned above, wherein the buffer comprises a compound which improves separation between capsids fully packaged with genetic material and capsids not fully packaged with genetic material. This compound may or may not be present in a buffer applied in step (a). Without being bound by theory, such a compound may for example improve separation by influencing interactions between capsid and ligand or interactions between capsid and capsid. Said compound which improves separation may for example be selected from a carbohydrate, a divalent metal ion, and a detergent.

Where said compound which improves separation is a carbohydrate, it may for example be selected from sucrose, sorbitol, and a polysaccharide.

Where said compound which improves separation is a divalent metal ion, it may for example be selected from Mg²⁺, Fe²⁺, and Mn²⁺. The metal ion may be present in the form of a salt, optionally in combination with for example chloride ions or sulphate ions. MgCl₂ is a non-limiting example of a suitable metal salt to include in the buffer of step (c), and step (b) when not fully packaged capsids are bound to the chromatography material. Non-limiting examples of suitable concentrations of MgCl₂ include from about 0.5 to about 30 mM of MgCl₂, such as from about 1 to about 20 mM, such as from about 2 to about 10 mM, or about 0.5, 1.0, 1.5, 2.0, 2.5, 3, 4, 5, 6, 7, 8, 9, 10, 11, 12, 13, 14, 15, 16, 17, 18, 19, 20, 25, or 30 mM, of MgCl₂. According to a non-limiting example, as described in the Example below, 2 mM MgCl₂ may be included in the buffer of steps (a)-(c).

Where said compound which improves separation is a detergent, it may for example be selected from poloxamer, such as poloxamer 188 or Pluronic™ F68, and polysorbate, such as Tween 20 or Tween 80.

In the above-described method, the adeno-associated virus capsids not fully packaged with genetic material may be obtained or eluted in step (b) by applying a buffer having a first value of conductivity or conductivity-related parameter and the adeno-associated virus capsids fully packaged with genetic material may be eluted in step (c) by applying a buffer having a second value of conductivity or conductivity-related parameter.

The first value of conductivity or conductivity-related parameter determined shall be suitable for eluting the adeno-associated virus capsids not fully packaged with genetic material, and the second value of conductivity or conductivity-related parameter determined shall be suitable for eluting the adeno-associated virus capsids fully packaged with genetic material. In a separation method where empty capsids are eluted before full capsids, a suitable first value of conductivity is normally the value of conductivity applied when eluting the first peak containing empty and/or full capsids. If the empty capsids do not bind to the column but end up in the flowthrough, the first value of conductivity will be the same as the baseline conductivity value, i.e., the conductivity before the step gradient of conductivity is applied. Accordingly, in some instances the first value of conductivity may be determined to be as low as 0 mS/cm.

In a separation method where empty capsids are eluted before full capsids, a suitable second value of conductivity is normally a value of conductivity equal to or higher than the conductivity value applied when eluting the last peak containing empty and/or full capsids. For example, if the last peak is eluted at a conductivity value of 5 mS/cm, the second value of conductivity is determined to be >5 mS/cm.

In the above-described method, step (c), and step (b) when not fully packaged capsids are bound to the chromatography material, may comprise applying a buffer, optionally one of the buffers mentioned above, wherein the buffer comprises a compound which may help eluting capsids bound to the chromatography material. This compound is not present in a buffer applied in step (a). If step (b) involves obtaining the not fully packaged capsids in a flow-through mode, this compound is neither present in step (b). Non-limiting examples of such a compound is a salt, such as a salt of a monovalent metal ion. More particularly, the salt may be a kosmotropic salt. Salts in water solvent are defined as kosmotropic (order-making) if they contribute to the stability and structure of water-water interactions. In contrast, chaotropic (disorder-making) salts have the opposite effect, disrupting water structure, increasing the solubility of nonpolar solvent particles, and destabilizing solute aggregates. Kosmotropes cause water molecules to favorably interact, which in effect stabilizes intramolecular interactions in macromolecules such as proteins (Moelbert S et al). A scale can be established for example by referring to the Hofmeister series, or lyotropic series, which is a classification of ions in order of their ability to salt out or salt in proteins (Hyde A et al).

More particularly, the kosmotropic salt may comprise (i) an anion selected from a group consisting of CO₃²⁻, SO₄²⁻, S₂O₃²⁻, H₂PO₄⁻, HPO₄²⁻, acetate⁻, citrate⁻, and Cl⁻, and (ii) a cation selected from a group consisting of NH₄⁺, K⁺, Na⁺, and Li⁺. In a currently preferred embodiment, the salt is sodium acetate (NaOAc). Non-limiting examples of suitable concentrations of NaOAc include from about 5 mM to about 500 mM, such as about 5, 10, 20, 30, 40, 50, 60, 70, 80, 90, 100, 150, 200, 250, 300, 350, 400, 450, or 500 mM. However, it is to be understood that other salts consisting of a combination an anion as listed under (i) and a cation as listed under (ii) may alternatively be used to elute the capsids. Non-limiting examples are NaCl, LiCl, KCl, or other equivalent metal salt suitable to use for salt elution, as is well known in the art. Non-limiting examples of suitable concentrations of NaCl include from about 5 mM to about 2M of NaCl, such as about 5, 10, 20, 30, 40, 50, 100, 150, 200, 250, 300, 350, 400, 450, 500, 600, 700, 800, 900, 1000, 1500, or 2000 mM, of NaCl. Further, step (b) may comprise applying a gradient of such a compound to improve elution of the adeno-associated virus capsids fully packaged with genetic material from the chromatography material. Such a gradient may be a linear gradient or a step gradient, or a combination thereof.

A non-limiting example of a suitable buffer to be applied in step (c), and in step (b) when not fully packaged capsids are bound to the chromatography material, may comprise 20 mM bis-Tris propane (BTP), pH 9.0, 2 mM MgCl₂, and 250 nM NaOAc.

The presently disclosed analytic separation method is applicable to adeno-associated virus capsids of different serotypes, such as capsids of adeno-associated virus serotype 1 (AAV1), adeno-associated virus serotype 2 (AAV2), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), or adeno-associated virus serotype 10 (AAV10), or a variant thereof. In a currently preferred embodiment of said method, the adeno-associated virus capsids are capsids of adeno-associated virus serotype 9 (AAV9) or a variant thereof.

According to a currently preferred embodiment, in the separation method as illustrated in FIG. 1, the chromatography material is defined by Formula IV:

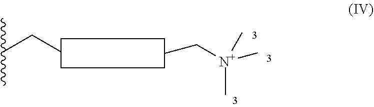

and the elution buffer of steps (b) and (c) comprises sodium acetate. Said method may be applied for separation of AAV capsids of any serotype or variant as described above. In particular, the capsids to be separated may be capsids of the AAV9 serotype or a variant thereof.

In the herein disclosed analytic method for separating adeno-associated virus capsids fully packaged with genetic material from adeno-associated virus capsids not fully packaged with genetic material, the chromatography material used may advantageously be a polishing chromatography material, meaning that the chromatography material is applied in a polishing step.

The term “polishing step” refers in the context of liquid chromatography to a final purification step, wherein trace impurities are removed to leave an active, safe product. Impurities removed during the polishing step are often conformers of the target molecule, i.e., forms of the target molecule having particular molecular conformations, or suspected leakage products. A polishing step may alternatively be called “secondary purification step”.

Further, the liquid sample added in step (a) of the herein disclosed method for separating adeno-associated virus capsids fully packaged with genetic material from adeno-associated virus capsids not fully packaged with genetic material may advantageously be a pre-purified liquid sample.

A pre-purified liquid sample comprising adeno-associated virus capsids, may be obtained by subjecting an adeno-associated virus capsid-containing cell culture harvest to a pre-purifying step, alternatively called a “capture step”. Most commonly, a capture step includes clarification (e.g., by filtration, centrifugation, or precipitation), and normally also concentration and/or stabilisation of the sample, and a significant purification from soluble impurities, for example by applying chromatography after the clarification, concentration, and stabilisation of sample. After the capture step, an intermediate purification may follow, which further reduces remaining amounts of impurities such as host cell proteins, DNA, viruses, endotoxins, nutrients, components of a cell culture medium, such as antifoam agents and antibiotics, and product-related impurities, such as aggregates, misfolded species, and aggregates. A pre-purifying step may include performing one or more of the following non-limiting examples of purification methods:
(i) affinity chromatography,
(ii) ion exchange chromatography,
(iii) precipitation or tangential flow filtration (TFF), followed by size-exclusion chromatography, such as by use of for example Capto Core 400 chromatography material (Cytiva, Sweden), which combines flow-through of the capsids with binding of impurities to the chromatography material,
(iv) TFF followed by ion exchange chromatography, and
(v) TFF followed by ion exchange chromatography and Capto Core.

Herein, the term “cell culture” refers to a culture of cells or a group of cells being cultivated, wherein the cells may be any type of cells, such as bacterial cells, viral cells, fungal cells, insect cells, or mammalian cells. A cell culture may be unclarified, i.e., comprising cells, or may be cell-depleted, i.e., a culture comprising no or few cells but comprising biomolecules released from the cells before removing the cells. Further, an unclarified cell culture may comprise intact cells, disrupted cells, a cell homogenate, and/or a cell lysate.

The term “cell culture harvest” is used herein to denote a cell culture which has been harvested and removed from the vessel or equipment, in which the cells have been cultivated.

The present disclosure further provides a kit of parts for analytical separation of adeno-associated virus capsids fully packaged with genetic material from adeno-associated virus capsids not fully packaged with genetic material present in a liquid sample, the kit of parts comprising the herein disclosed chromatography device as described in detail elsewhere herein, and one or more buffers.

A currently preferred chromatography device to include in a kit of parts is a 1 mL HiTrap Capto Q column (Cytiva, Sweden).

Said buffers are selected from the buffers described in detail elsewhere herein. In a currently preferred embodiment, the kit of parts comprises a buffer system containing a buffer A containing 20 mM BTP pH 9.0 and 2 mM MgCl₂, and a buffer B containing 20 mM BTP pH 9.0, 2 mM MgCl₂ and 250 mM NaOAc. The kit of parts may further contain written instructions for use of said buffers.

The kit of parts may optionally also comprise a computer program product directly loadable into the internal memory of a digital computer, which computer program product comprises software code means for translation of UV signal ratio into viral capsids per mL. The kit of parts may further contain written instructions for use of said computer program product.

A non-limiting example of a computer program product which may be included in the kit of parts is a computer program product based on a commercially available Unicorn evaluation software, which may be used with Äkta™ pure 25 chromatography systems (Cytiva, Sweden).

The present disclosure is also directed to a chromatography system comprising:
a. a sample loop having a volume of from about 10 μL to about 2 mL, such as a volume of about 10 μL, 20 μL, 30 μL, 40 μL, 50 μL, 60 μL, 70 μL, 80 μL, 90 μL, 100 μL, 200 μL, 300 μL, 400 μL, 500 μL, 600 μL, 700 μL, 800 μL, 900 μL, 1 mL, 1.25 mL, 1.5 mL, 1.75 mL, or 2 mL;
b. the herein disclosed chromatography device as described in detail elsewhere herein;
c. a detector for measuring absorbance, optionally for measuring absorbance at 260 nm and 280 nm;
d. a detector for measuring fluorescence, optionally for measuring excitation at 280 nm and emission at 348 nm;
e. a detector for measuring multi-angle light scattering, optionally at an angle of 90°; and
f. pumps for pumping flows and volumes relevant for analytic separation of adeno-associated virus capsids fully packaged with genetic material from adeno-associated virus capsids not fully packaged with genetic material present in a liquid sample.

Sample loops, detectors for measuring absorbance, fluorescence, and multi-angle light scattering, as well as pumps for pumping flows and volumes relevant for analytic separation of AAV capsids are well known in the art and can easily be chosen by the skilled person.

Non-limiting examples of a currently available chromatography system which is suitable for application with the herein disclosed chromatography device are Äkta™ pure 25 chromatography systems (Cytiva, Sweden).

Another non-limiting example of a currently available chromatography system which is suitable for application with the herein disclosed chromatography device is a Bio-inert HPLC system (Agilent, USA).

The chromatography system may optionally also comprise a computer program product directly loadable into the internal memory of a digital computer, which computer program product comprises software code means for translation of UV signal ratio into viral capsids per mL.

A non-limiting example of a computer program product which may be included in the chromatography system is a computer program product based on a commercially available Unicorn evaluation software, which may be used with Äkta™ pure 25 chromatography systems (Cytiva, Sweden).

Devices or compositions “comprising” one or more recited components may also include other components not specifically recited. The term “comprising” includes as a subset “consisting essentially of” which means that the device or composition has the components listed without other features or components being present. Likewise, methods “comprising” one or more recited steps may also include other steps not specifically recited.

The singular “a” and “an” shall be construed as including also the plural.

### Example 1: Analytic Separation of Capsids of Serotypes AAV8 and AAV9 on Anion Exchange Chromatography Material Using Magnesium Chloride and Sodium Acetate Step Gradient

### Chromatography Material

The following currently available anion exchange chromatography material gave improved results compared to prior art chromatography materials, as described further below:

### Capto Q (Cytiva, Sweden):

Ligand: Quaternary amine
Particle size, d₅₀ᵥ: ˜90 μm
Matrix: Highly cross-linked agarose with dextran surface extender
Ionic Capacity: 0.16-0.22 mmol Cl⁻/ml medium
pH stability, operational: 2-12

### Equipment and Samples

The analytical anion exchange resin was packed in a 1 mL HiTrap column according to the packing instructions. The runs were performed using an Äkta Pure P25 system with a flowrate of 5 mL/min, with the mixer of the system disconnected in order to minimize the dead volume and to get sharp conductivity steps. The sample was applied to the previously equilibrated column using a capillary loop. Typically, samples applied to the resin comprised affinity purified, or affinity and size exclusion purified, AAV8 or AAV9, respectively, at a concentration of approx. 10¹⁰ AAV capsids, containing a mixture of full and empty capsids (approx. 40-50% full capsids based on qPCR:ELISA ratio). Each AAV sample was prepared to have a conductivity of approximately <3 mS/cm, either by a 10-50 fold dilution, or by buffer exchange, before loading the sample onto the analytical anion exchange resin.

The 280 and 260 nm UV absorbance were monitored during the runs and the 260/280 ratios were used as a diagnostic tool to navigate in the chromatogram and distinguish between full and empty capsid populations. The chromatograms were analyzed using the Evaluation package of Unicorn. A 260/280 ratio above 1.2 is considered to indicate 100% full capsids, and a 260/280 ratio of approx. or below 0.6-0.7 is considered to indicate 100% empty capsids.

### Process Conditions and Results

The currently available anion exchange resin Capto Q with dextran extenders was evaluated by applying a two-step elution method using a buffer system including a buffer A and a buffer B, both containing 20 mM Bis-Tris Propane (BTP) pH 9.0 and 2 mM MgCl2, and buffer B additionally comprising 250 mM sodium acetate (NaOAc) as elution salt.

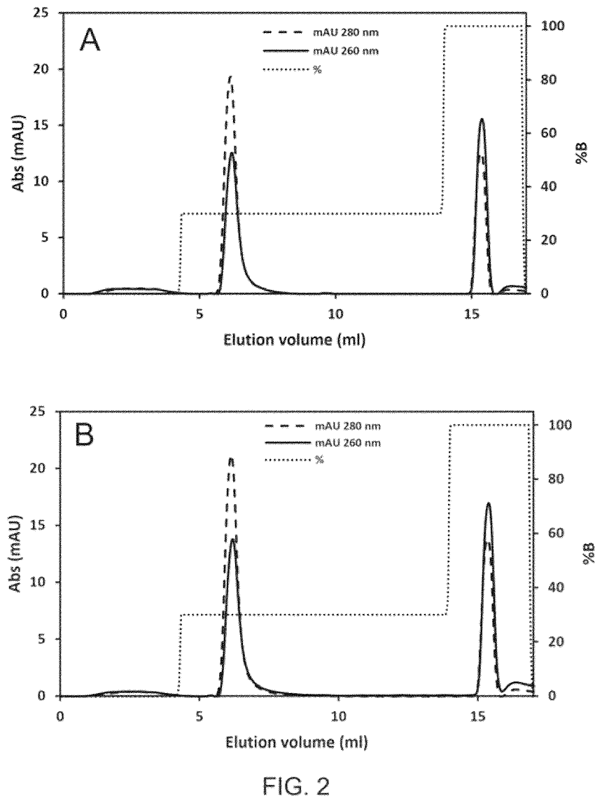

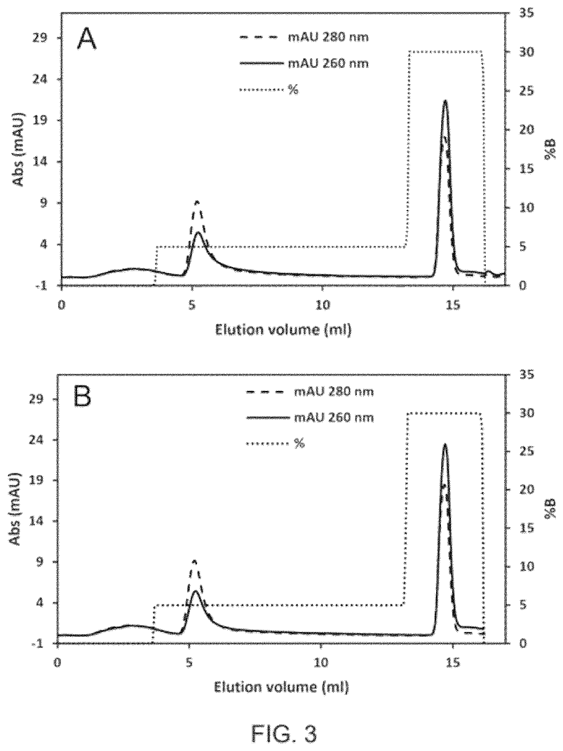

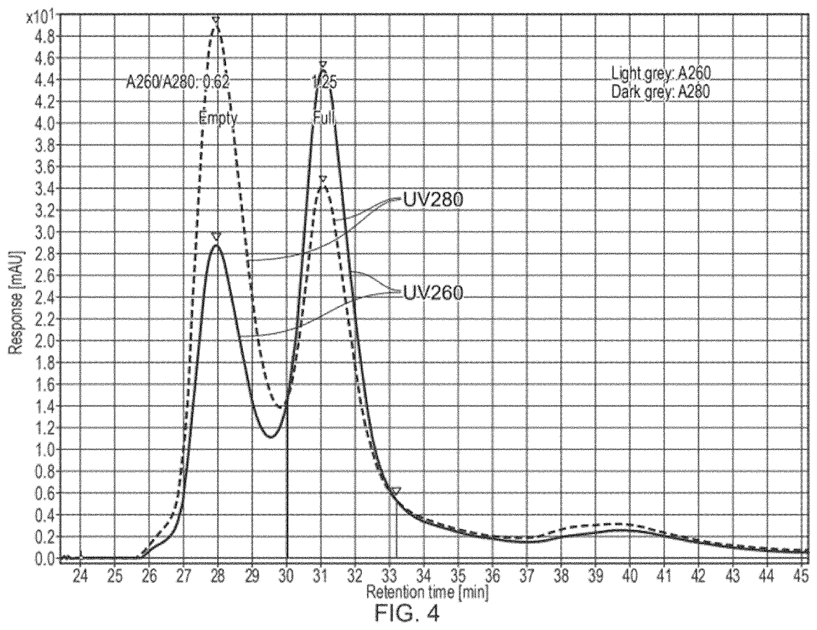

In FIGS. 2-3, the y-axis on the right-hand side of each chromatogram denotes the percentage of buffer B included in the resulting elution buffer (the rest being buffer A) during elution from the chromatography material.
Equilibration: 1 CV buffer A
Injection: 2 mL AAV8 or AAV9 (approx E10 VP/mL)
Wash: 1 CV buffer A
Two-step elution: The conditions applied for each serotype are specified below
Re-equilibration: 1 CV buffer A
AAV8: Step 1 30% B, 10 CV
   Step 2 100% B, 3 CV
AAV9: Step 1 5% B, 10 CV
   Step 2 30% B, 3 CV

Both tested serotypes (AAV8 and AAV9) resulted in a good separation of full and empty capsids (FIGS. 2-3). Elution of full (F) and empty (E) capsids, and UV260:280 ratios are indicated in FIGS. 2-3. FIGS. 2A-B show the chromatograms for duplicate runs of the NaOAc two-step elution of AAV8. FIGS. 3A-B show the chromatograms for duplicate runs of the NaOAc two-step elution of AAV9.

The total run time for each run was only 3.5 min.

### Comparative Data for Previously Known Analytical Chromatography Material

Previously known CIMac™ AAV full/empty-0.1 Analytical Column (a monolithic column) and CIMmultus™ QA 1 mL Monolithic Column (both from Sartorius, Germany), were evaluated under the following conditions:

### CIMmultus™ QA 1 mL Monolithic Column:

HPLC system (Agilent)
Injection: 30 μL AAV8 sample (approx. E11 VP mL)
Detection: MWD, overlay
Flow: 1 CV/min
   Buffer system:
A: 20 mM BTP pH 9.5, 2 mM MgCl2, 1% sucrose, 0.1% Poloxamer 188
B: Buffer A+400 mM NaCl
Gradient: 0-100% B, 0-100 min

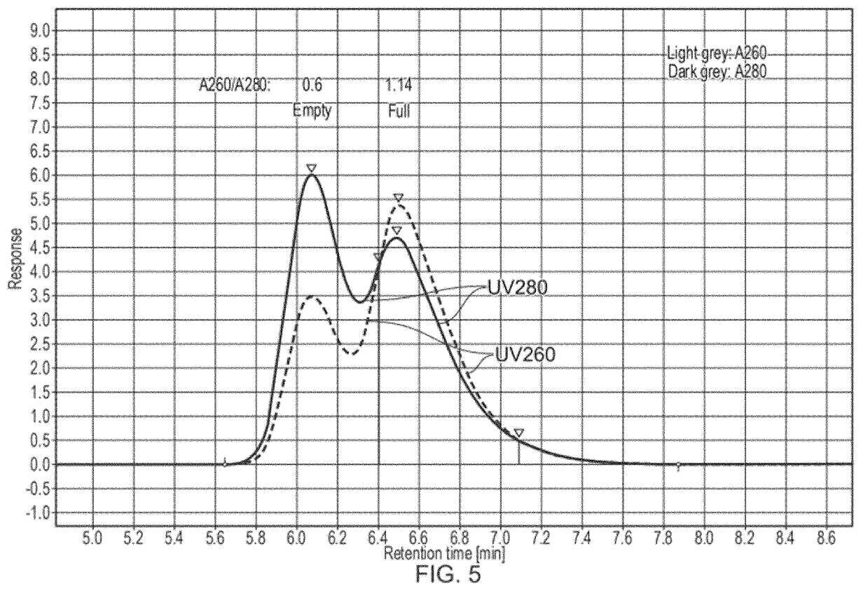

This chromatography material resulted in a separation of full and empty AAV8 capsids with overlapping peaks and did not achieve a good baseline separation of full and empty AAV8 capsids, as seen in FIG. 4. The x-axis of FIG. 4 denotes the retention time in minutes and the y-axis of FIG. 4 denotes the absorbance response in mAU.

### CIMac™, 0.1 mL Analytical Column:

HPLC system (Agilent)
Injection: 50 μL AAV5 sample (37% full capsids)
Detection: MWD, overlay
Flow: 1 mL/min
   Buffer system:
A: 20 mM BTP pH 9.5, 2 mM MgCl₂, 1% sucrose, 0.1% Poloxamer 188
B: Buffer A+400 mM NaCl
Gradient: 0-100% B, 0-20 min

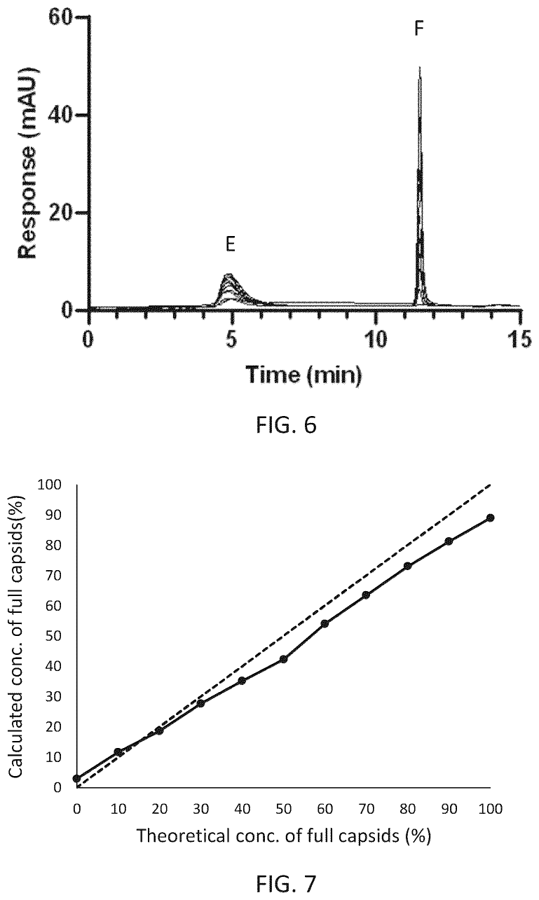

This chromatography material resulted in a separation of full and empty AAV5 capsids with overlapping peaks and did not achieve a good baseline separation of full and empty AAV5 capsids, as seen in FIG. 5. The x-axis of FIG. 5 denotes the retention time in minutes and the y-axis of FIG. 5 denotes the absorbance response in mAU.

The total run time for the previously known chromatography devices was significantly longer than the total run time for the herein disclosed chromatography devices.

### Example 2: Analytic Separation of AAV8 Samples of Various Empty:Full Capsid Ratios on Anion Exchange Chromatography Material

A preparative Capto Q chromatography material (see Example 1) separating empty and full capsids from affinity purified AAV8 was run. Peak 1 (empty) and peak 2 (full) fractions were pooled and full and empty capsid ratio was determined by qPCR/ELISA. Full and empty capsid samples were then mixed according to Table 1. Each prepared sample volume was 150 μl.

TABLE 1
Preparation of samples at different ratios
of AAV8 full (F) and empty (E) capsids.

| Sample | Volume empty capsid | Volume full capsid |
|---|---|---|
| ratio E:F | fraction (μl) | fraction (μl) |
| 0:10 | 0 | 150 |
| 10:90 | 15 | 135 |
| 20:80 | 30 | 120 |
| 30:70 | 45 | 105 |
| 40:60 | 60 | 90 |
| 50:50 | 75 | 75 |
| 60:40 | 90 | 60 |
| 70:30 | 105 | 45 |
| 80:20 | 120 | 30 |
| 90:10 | 135 | 15 |
| 100:0 | 150 | 0 |

Next, 100 μl of each sample containing approximately 10¹⁰ to 10¹¹ viral particles (VP) was subjected to chromatographic separation on a 0.5 mL Tricorn 5/20 Capto Q column (Cytiva, Sweden) with a 1260 Infinity LC system (Agilent, USA) using the protocol outlined in Table 2. Flow rate was 1 mL/minute for all steps. Buffer A: 20 mM Tris-Cl, 2 mM MgCl₂. Buffer B: 20 mM Tris-Cl, 2 mM MgCl₂, 250 mM NaCl. The % buffer B for step 1 and 2 elution was determined by prescreening and changes in UV260:280 as described in international patent application PCT/EP2023/060871. UV was measured at 260 nm and 280 nm. Fluorescence was measured through 280 nm excitation and 350 nm emission.

TABLE 2

| Step | Buffer | Volume | |
|---|---|---|---|
| Injection | | 100 | μl |
| Column wash | A-buffer | 5 | CV |
| Elution 29% | A-buffer with 29% B-buffer | 15 | CV |
| Elution 50% | A-buffer with 50% B-buffer | 5 | CV |
| Elution 100% | B-buffer | 5 | CV |
| CIP 1 | 1M NaOH | 10 | CV |
| CIP 2 | 2M NaCl | 10 | CV |
| Equilibration | A-buffer | 10 | CV |

FIG. 6 shows an overlay of the analytical anion exchange separation chromatograms from AAV8 full and empty capsid mixes containing 10, 20, 30, 40, 50, 60, 70, 80, 90 and 100% full capsids. Calculation of % full capsids in each sample mix was based on peak areas from the first empty capsid peak and the second full capsid peak. In FIG. 7 the calculated concentration of AAV8 full capsids based on peak areas (solid line) was plotted against the theoretical concentration (dotted line) of full capsids in the prepared AAV8 sample mixes (from qPCR and ELISA results).

It is to be understood that the present disclosure is not restricted to the above-described exemplifying embodiments thereof and that several conceivable modifications of the present disclosure are possible within the scope of the following claims.

### REFERENCES

Weihong Qu et al, Scalable Downstream Strategies for Purification of Recombinant Adeno-Associated Virus Vectors in Light of the Properties, Current Pharmaceutical Biotechnology 2015 August; 16(8): 684-695
Xiaotong Fu et al, Analytical Strategies for Quantification of Adeno-Associated Virus Empty Capsids to Support Process Development, Human gene therapy methods, 2019, 30(4): 144-152
Moelbert Susanne et al, Kosmotropes and chaotropes: modelling preferential exclusion, binding and aggregate stability, Biophysical Chemistry, 2004 December, 112(1): 45-57
Hyde Adam M et al, General Principles and Strategies for Salting-Out Informed by the Hofmeister Series, Organic Process Research & Development, 2017, 21 (9): 1355-1370.

1. A chromatography device for use in an analytic separation method, comprising a chromatography material, which comprises a support and a ligand, wherein the ligand of the chromatography material is defined by the following Formula I: 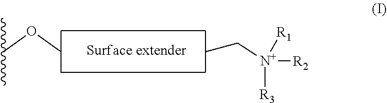
wherein R₁ is selected from C1-C3 alkyl, and R₂ and R₃ are independently selected from C1-C3 alkyl, CH2OH, and CH2CHOHCH3,
wherein the volume of the chromatography material is from about 0.1 mL to about 2 mL.

2. The chromatography device of claim 1, wherein the volume of the chromatography material is from about 0.1 mL to about 1.5 mL, such as about 0.1 mL to about 1.0 mL or about 0.1 mL to about 0.5 mL.

3. The chromatography device according to claim 1, wherein the chromatography material has a capacity of separating from about 10¹⁰ to at least about 10¹³ virus capsids from a liquid sample having a volume of from about 70 μL to about 2 mL.

4. The chromatography device according to claim 1, wherein each of R₁, R₂, and R₃ is CH3.

5. The chromatography device according to claim 1, wherein the surface extender is a polymer, wherein the polymer is selected from:
(i) a polymer having a naturally occurring skeleton, such as a polysaccharide, such as starch, cellulose, dextran, or agarose, and
(ii) a polymer having a synthetic skeleton, such as a polyvinyl alcohol, a polyacrylamide, a polymethacrylamide, or a polyvinyl ether;
preferably wherein the surface extender is dextran, optionally wherein the density of dextran is from about 5 to about 30 mg dextran per mL of the chromatography material.

6. Use of the chromatography device according to claim 1, for analytic separation of adeno-associated virus capsids fully packaged with genetic material from adeno-associated virus capsids not fully packaged with genetic material present in a liquid sample, wherein the chromatography material has a capacity of separating from about 10¹⁰ to at least about 10¹³ virus capsids, wherein the volume of the liquid sample is from about 70 μL to about 2 mL.

7. The use according to claim 6, wherein the minimum time for the liquid sample to pass through the chromatography material is from about 2 minutes to about 5.5 minutes.

8. The use according to claim 6, wherein the adeno-associated virus capsids are capsids of adeno-associated virus serotype 1 (AAV1), adeno-associated virus serotype 2 (AAV2), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), or adeno-associated virus serotype 10 (AAV10), or a variant thereof;
optionally wherein the adeno-associated virus capsids are capsids of adeno-associated virus serotype 9 (AAV9) or a variant thereof.

9. A method for separating adeno-associated virus capsids fully packaged with genetic material from adeno-associated virus capsids not fully packaged with genetic material present in a liquid sample, comprising the following steps:
a. adding a liquid sample comprising adeno-associated virus capsids of a purity of at least 90% and of a concentration of at least 10¹⁰ adeno-associated virus capsids/mL, of which at least 10% of the adeno-associated virus capsids are adeno-associated virus capsids fully packaged with genetic material, to a chromatography material comprised by the chromatography device of claim 1;
b. obtaining the adeno-associated virus capsids not fully packaged with genetic material in a flow-through mode, or eluting the adeno-associated virus capsids not fully packaged with genetic material from the chromatography material;
c. eluting the adeno-associated virus capsids fully packaged with genetic material from the chromatography material;
wherein the adeno-associated virus capsids eluted in step (c) are eluted into eluate fractions, which eluate fractions combined comprise at least 80% of the adeno-associated virus capsids of the liquid sample added in step (a), of which at least 90% of the adeno-associated virus capsids are fully packaged with genetic material.

10. The method according to claim 9, wherein the volume of the liquid sample added in step (a) is from about 70 μL to about 2 mL.

11. The method according to claim 9, wherein steps (a)-(c) and any intermediate steps can be completed within 5.5 minutes.

12. The method according to claim 9, wherein the adeno-associated virus capsids not fully packaged with genetic material are obtained or eluted in step (b) by applying a buffer having a first value of conductivity or conductivity-related parameter and the adeno-associated virus capsids fully packaged with genetic material are eluted in step (c) by applying a buffer having a second value of conductivity or conductivity-related parameter.

13. The method according to claim 9, wherein step (c), and optionally step (b), comprises applying a buffer comprising a kosmotropic salt,
wherein the salt comprises (i) an anion selected from a group consisting of CO₃²⁻, SO₄²⁻, S₂O₃²⁻, H₂PO₄⁻, HPO₄²⁻, acetate⁻, citrate⁻, and Cl⁻, and (ii) a cation selected from a group consisting of NH₄⁺, K⁺, Na⁺, and Li⁺; optionally wherein the salt is sodium acetate.

14. The method according to claim 9, wherein
steps (b) and (c), and optionally step (a), comprise applying a buffer comprising a compound which improves separation between capsids fully packaged with genetic material and capsids not fully packaged with genetic material, optionally wherein said compound is selected from a carbohydrate, a divalent metal ion, and a detergent;
optionally wherein the carbohydrate is selected from sucrose, sorbitol, and a polysaccharide;
optionally wherein the divalent metal ion is selected from Mg²⁺, Fe²⁺, and Mn²⁺, optionally wherein the divalent metal ion is present in the form of a salt, optionally in combination with chloride ions or sulphate ions;
optionally wherein the detergent is selected from poloxamer and polysorbate.

15. The method according to claim 9, wherein the adeno-associated virus capsids are capsids of adeno-associated virus serotype 1 (AAV1), adeno-associated virus serotype 2 (AAV2), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), or adeno-associated virus serotype 10 (AAV10), or a variant thereof;
optionally wherein the adeno-associated virus capsids are capsids of adeno-associated virus serotype 9 (AAV9) or a variant thereof.

16. The method according to claim 9, wherein the chromatography material is defined by Formula IV: 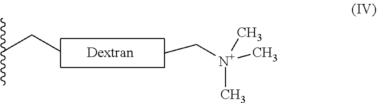
and wherein the buffer of steps (b) and (c) comprises sodium acetate;
optionally wherein the adeno-associated virus capsids are capsids of adeno-associated virus serotype 9 (AAV9) or a variant thereof.

17. A kit of parts for analytical separation of adeno-associated virus capsids fully packaged with genetic material from adeno-associated virus capsids not fully packaged with genetic material present in a liquid sample, the kit of parts comprising the chromatography device according to claim 1, and one or more buffers.

18. The kit of parts according to claim 17, further comprising a computer program product directly loadable into the internal memory of a digital computer, which computer program product comprises software code means for translation of UV signal ratio into viral capsids per mL.

19. A chromatography system comprising:
a. a sample loop having a volume of from about 10 μL to about 2 mL;
b. the chromatography device according to claim 1;
c. a detector for measuring absorbance, optionally for measuring absorbance at 260 nm and 280 nm;
d. a detector for measuring fluorescence, optionally for measuring excitation at 280 nm and emission at 348 nm;
e. a detector for measuring multi-angle light scattering, optionally at an angle of 90°; and
f. pumps for pumping flows and volumes relevant for analytic separation of adeno-associated virus capsids fully packaged with genetic material from adeno-associated virus capsids not fully packaged with genetic material present in a liquid sample.

20. The chromatography system according to claim 19, further comprising a computer program product directly loadable into the internal memory of a digital computer, which computer program product comprises software code means for translation of UV signal ratio into viral capsids per mL.
